(19) United States
(12) Patent Application Publication
(10) Pub. No.: US 2006/0233878 A1
(43) Pub. Date: Oct. 19, 2006

(54) Extended release formulation of beta-lactam antibiotics

(75) Inventors: Shailesh Bhamare, Pune (IN); Indu Bhushan, Pune (IN); Himadri Sen, Pune (IN)

Correspondence Address: VOLPE AND KOENIG, P.C., UNITED PLAZA, SUITE 1600, 30 SOUTH 17TH STREET, PHILADELPHIA, PA, 19103, US

(73) Assignee: Lupin Limited, Mumbai, Maharashtra (IN)

(21) Appl. No.: 10/568,325

(22) PCT Filed: Sep. 30, 2003
(86) PCT No.: PCT/IN03/00326

§ 371 Date: Feb. 16, 2006

(51) Int. Cl. A61K 31/545; A61K 9/22

(52) U.S. Cl. 424/468; 514/200

(57) ABSTRACT

A pharmaceutical composition for controlled drug delivery comprising a β-lactam antibiotic or its pharmaceutically acceptable hydrates, salts or esters, and one or more carbomers. The above β-lactam antibiotics formulation avoids the limitations of known β-lactam controlled release form which are found to be either complex and/or cost-extensive to obtain requiring multiphase and/or selective coatings or fail to achieve the desired controlled release for once daily dosage form. Importantly, in the β-lactam antibiotic form of the above the rate-controlling polymer wherein the Cₘₐₓ of the formulation is substantially the same as that of a single dose of the immediate release formulation. Also advantageously the formulation achieves a rate controlling polymer wherein the T>MIC for the formulation is more than 17 hours when the MIC is 0.25 mcg/ml and more than 10 hours when the MIC is 2 mcg/ml. The above β-lactam antibiotic form is thus directed to serve as the much desired simple and cost-effective controlled release form suitable for once daily administration.

### FIELD OF INVENTION

This invention relates to novel controlled release oral drug delivery system for β-lactam antibiotic agents and to its process of manufacture.

### BACK GROUND OF INVENTION

To enable optimal design of controlled release systems, a thorough understanding of pharmacokinetics and pharmacodynamics of the drug is necessary. Drug concentration in plasma is no more than a “surrogate” for pharmacological and clinical effects, the relevance of which can only be judged if the relationship between pharmacokinetics and pharmacodynamics (PK/PD) is well established.

Historically, pharmacokinetic properties of a drug are well understood and considered in designing of a controlled release dosage form. However, the pharmacodynamic aspects are rarely a factor in development of a drug delivery system. Absence of linear or direct relationship between plasma concentration of the drug and the magnitude of pharmacological response compromise the efficacy of drug delivery system. In the case of antimicrobial agents, this relationship depends on three elements: the pathogen, the host and the specific antimicrobial agent. The impact of the host, apart from the pharmacokinetic properties depends mainly on its immune system. The relationship between drug concentration and its inhibitory effects on microbial growth for a certain drug pathogen combination can be determined in vitro. The extrapolation of the in vitro data to an in vivo situation is less complex when the pathogen is located extracellularly, as in the case of β-lactam susceptible microorganisms.

In assessing the properties of β-lactam antibiotics in light of the principles outlined above, it has been concluded that an oral controlled-release preparation that would maintain low but effective concentrations for a prolonged period would be the suitable mode of administration of these medications. This conclusion is based on the following points: (1) The biological half-life of these agents is considerably short (about 1-2 hrs), which necessitates frequent administration; (2) Elevation of the drug concentration above the minimal inhibitory concentration (MIC) is not associated with increased bacteriocidal potency; (3) There is a direct correlation between the time above MIC and antimicrobial potency. There is no correlation between Area Under Curve (AUC) values and the drug's efficacy; (4) It has been confirmed that continuous infusion is advantageous to periodic bolus administration of these agents; (5) For these drugs there is a minimal effective concentration before the bacteriocidal effect is noted; (6) With the single exception of penem antibiotics, all the P-lactams exhibit either no post antibiotic effect (PAE) or a very short PAE; (7) High concentrations are associated with reduced potency; (8) The penetration of the drug into the tissues is not correlated to the serum concentration, i.e., elevation of serum drug concentrations will not contribute much in case where the pathogen is located intracellularly; (9) Unlike aminoglycosides, the kinetics of the bactericidal effect are slow and require maintenance of drug effective concentration for a certain lag time to the onset of effect.

Major parameters used to qualify the effect of anti-microbial drugs are minimum inhibitory concentration (MIC) and minimum bactericidal concentration (MBC), total concentration of drug in body and time over MIC.

Craig et al have reported Time over MIC (T>MIC) as surrogate end point/marker for measurement of therapeutic effect of P-lactam antibiotics. He indicated T>MIC more than 40% of dosing interval is required to achieve 80-90% of bacteriological efficacy.

From the above it is evident that any drug delivery system comprising of β lactain antibiotics should maintain the drug concentration in blood above MIC for more than 40% of dosing interval so as to achieve the desired clinical effect. Hence conversion of dosage regimen of β lactam antibiotics from TID/BID to OD would require to control delivery of drug in blood to maintain the blood concentration above MIC for prolonged period sufficient to achieve T>MIC for more than 40% of dosing interval.

Several attempts have been made to develop controlled release formulations for β-lactam antibiotics.

U.S. Pat. No. 4,250,166 discloses a long-acting cephalexin preparation comprising of normal quick-releasing cephalexin and particulate cephalexin coated with a copolymer of methylmethacrylate and methacrylic acid which dissolves at a pH from 5.5 to 6.5 and the potency ratio of the normal cephalexin to coated cephalexin is between 40:60 and 25:75.

U.S. Pat. No. 4,713,247 discloses a long-acting cefaclor formulation comprising of a mixture of non-enteric coated rapid-release cefaclor component and an enteric coated slow-release cefaclor component at a ratio of 4:6 based upon cefaclor potency, wherein the rapid-release component releases the drug in gastric fluid while the slow-release component dissolves at pH 5 to 7, thereby enabling oral administration thereof twice a day.

U.S. Pat. No. 4,968,508 discloses a sustained release matrix tablet comprising from about 0.1% to about 90% by weight of cefaclor, about 5% to about 29% by weight of hydrophilic polymer and about 0.5% to about 25% by weight of an acrylic polymer which dissolves at a pH in tle range of about 5.0 to about 7.4, tile total Veight of polymers being less than 30% by weight of the formulation. Although a specific cefaclor formulation is claimed, the text suggests that the matrix formulation is suitable for weakly basic drugs and particularly suitable for cephalexin and cefaclor.

U.S. Pat. No. 5,948,440 discloses a controlled release tablet of an active ingredient compromising of cefaclor, cephalexin; or their pharmaceutically acceptable hydrates, salts, or esters as active ingredient, and a mixture of hydrophilic polymers selected from the group consisting of at least one hydroxypropyl methylcellulose and at least one hydroxypropylcellulose. The composition optionally also contains one or more of a water-soluble or water dispersible diluent. The quantities of the hydrophilic polymers and water-soluble or water dispersible diluent are such that the therapeutically effective active ingredient is released at a rate suitable for twice daily administration of the pharmaceutical composition.

Japanese Patent JP 57165392A discloses a long-acting cephalexin tablet comprising cephalexin mixed with ≧10% w/w oils and fats (e.g. higher fata acid, higher alcohol, alcohol ester, etc.) and with a vehicle such as microcrystalline cellulose and a lubricant such as magnesium stearate, and the mixture is pressed, formed to granules passing through a 20 mesh sieve, and subjected to the slug-forming process to obtain a high-quality long-acting tablet. The rate of dissolution of cephalexin can be controlled by selecting the kind of oils and fats and the number of the times of slug formation process.

Japanese Patent JP 07010758A discloses a long acting cefaclor composition comprising rapidly soluble cefaclor and a delayed soluble cefaclor prepared by enteric coating of hydroxypropyl methyl cellulose acetate succinate and triethyl citrate.

U.S. Pat. No. 6,399,086 discloses a controlled release β-lactam antibiotic agent preferably amoxicillin trihydiate in a hydrophilic and/or hydrophobic polymeric matrix such that 50% of the active is released within 3 to 4 hr from oral administration and remainder is released at a controlled rate.

Although controlled release formulations have been disclosed in the prior art, none of them have been studied for their pharmacodynamic properties on which the efficacy of these dosage forms would be dependent. Further, most of these patents describe formulations involving the use of multiple polymers for controlling the rate of drug release.

An orally controlled drug delivery system of β-lactam antibiotics encounters with the typical physiochemical properties such as amphoteric behavior, having isoelectric point, pH dependent solubility and pH dependent stability. Due to the unique nature of pH dependent solubility of β-lactam antibiotics, majority of common polymer such as HPMC, HPC, Xanthan Gum, Alginates, Gaur Gum when used as release controlling agent in matrix dosage form, release drug at faster rate in acidic pH and at slower rate in alkaline pH. Further exponential release of drug from matrix system contributes to enhance the drug release during initial stage, leading to bursting effect.

Some investigators have tried to overcome these limitations in the prior art by using combination of polymers having pH dependent and pH independent solubility such as sodium alginate that is soluble above pH 5 in combination with xanthan gum.

But the use of such polymers and/or their combinations have their own limitation of dissolution stability leading to increase in dissolution rate on storage at accelerated and long-term storage conditions.

Accordingly, the oral controlled drug delivery systems of β-lactam antibiotic of the known art are either complex and cost-extensive to obtain requiring multiphase and/or selective coatings or fail to achieve the desired controlled release.

### OBJECTS OF THE INVENTION

It is an object of the present invention to provide a pharmaceutical composition for controlled release of β-lactam antibiotics which would avoid the above discussed limitations of such β-lactam controlled release form and would be also effective as a once daily dosage form.

It is another object of the present invention to provide a pharmaceutical composition for controlled release of P-lactam antibiotics comprising the active ingredient, and one or more carbomers.

It is a further object of the present invention to provide a controlled release formulation comprising a β-lactam antibiotic and a rate-controlling polymer wherein the Cₘₐₓ of the formulation is substantially the same as that of a single dose of the immediate release formulation.

It is another object of the present invention to provide a controlled release formulation comprising a β-lactam antibiotic and a rate controlling polymer wherein the T>MIC for the formulation is more than 17 hours when the MIC is 0.25 mcg/ml and more than 10 hours when the MIC is 2 mcg/ml.

It is a further object of the present invention to provide a process for the preparation of the controlled release formulation comprising the β-lactam antibiotic and one or more acrylic acid polymers, optionally with one or more diluents and lubricants and compressing them to tablets either directly or after dry compaction to get granules.

It is yet another object of the present invention to provide a controlled release composition comprising from about 30-90% w/w of cefprozil and from about 0.1-50% by weight of one or a mixture of carbomers and optionally one or more pharmaceutically acceptable excipients selected from amongst diluents and lubricants

### SUMMARY OF INVENTION

The present invention provides a novel pharmaceutical composition for controlled drug delivery comprising a β-lactam antibiotic or its pharmaceutically acceptable hydrates, salts or esters, and one or more carbomers. Optionally the composition also contains one or more water soluble and/or water dispersible diluent, wherein the quantities of acrylic acid polymer and water soluble and/or water dispersible diluent are Such that the β-lactam antibiotic is released at a controlled rate suitable over an extended period of time.

### DETAILED DESCRIPTION OF THE INVENTION

In the above controlled release formulation of the present invention, β-lactam antibiotics are selected from amongst cephalosporins or their pharmaceutically acceptable hydrates, salts or esters.

The cephalosporins are selected from amongst cefdinir, cefditoren piroxil, cefepime, cefixime, cefoperazone, cefotetan, cefoxitin, cefpodoxime paroxetil, cefprozil, cefazidine, ceftibuten, ceftriaxone, cefuroxime axetil, cephaixin, cefaclor, cefadroxil, cefamandole, cefoxitin, cephalothin, moxalactum, cephapirin, ceftizoxime, cefonicid, cefadrine, loracarbef and the like or their pharmaceutically acceptable hydrates, salts or esters thereof.

In the more preferred embodiment of the present invention, the cephalosporin is cefprozil or its pharmaceutically acceptable hydrates, salts or esters. It is present in an amount from about 30% to about 90% by weight of the controlled release matrix.

Further, the cefprozil or their pharmaceutically acceptable hydrates; salts or esters may be present in an amount from 100 mg to 1000 mg per dosage form.

According to present invention, the pharmaceutical composition contains one or more acrylic acid polymers. In a preferred embodiment of the present invention, the acrylic acid polymer essentially consists of one or a mixture of carbomers. (manufactured by B.F. Goodrich, USA under the trade name ‘Carbopol’)

Carbomers are acrylic acid polymers, cross-linked with polyalkenyl ethers making it soluble in water. Since the p^{Ka} of these polymers is 6.0±0.5, the carboxylate groups on the polymer backbone ionize to form gel by swelling due to repulsion between negative charges when exposed to a pH environment above 4.0-6.0. Thus, due to this semi enteric behavior of this polymer, it provides advantage over other polymers like HPMC, HPC, xanthan gum when used in formulation of controlled release matrix delivery of β-lactam antibiotic system by controlling the bursting effect of drug in initial stage of drug release in acidic media.

At low pH (5.0 or less), less than 10% of the carlbopol acid groups will be ionized resulting in relatively little swelling leading to hydrogen bonding to polysaccharide and protein as a major mechanism of bioadhesion to the mucin layer.

At higher pH, the carboxylic acid groups are ionized to a greater extent resulting in highly swollen gel formed by electrostatic repulsion of the anionic charges along with the backbone.

This reduces the hydrogen bonding but increases interaction of polycarboxylate with cationic (protonated or quaternary) bases and with polyvalent ions bound on protein or polysaccharides resulting in bioadhesion in alkaline pH.

Further, carbomers are found to be compatible with large number of β-lactam antibiotics compared to various polymers routinely used for preparation of controlled release dosage form. These polymers may be present at 0.1%-50% w/w of the composition. More preferably they are present from about 0.1 to about 40% w/w of the composition.

In the formulation of the present invention describes the use of a single carbomer or a mixture of various grades of carbomers can be made in order to modify the drug release from the matrix.

Carbopol 971P comprises few cross-link sites which opens up early at low concentration eliminating the interstitial space between the swollen gel particles producing “Fish net” gel structure upon hydration providing significant resistance to small diffusing molecules.

Carbopol 974P on the other hand comprises more cross link sites which does not open up easily producing interstitial space at lower concentration that act as channels for the release of drug at faster rate. This combination of the carbopol 971P and carbopol 974P can be manipulated to achieve the desired drug release profile.

In accordance wraith a preferred aspect, in the mix, carbopol 971P can be at 0.1-20% w/w of the controlled release formulation and carbopol 974P can be at 0.1-20% w/w of the formulation provided the total carbopol content is between 0.1-50%.

According to present invention, the pharmaceutical composition may further contain one or more of pharmaceutically acceptable excipients selected from amongst diluents, lubricants in an amount of about 1% to about 30% by weight. More preferably they are present from about 5 to about 25% w/w of the composition.

The diluents may be water-soluble or water dispersible. Examples of water-soluble diluents that may be used in the present invention include lactose, mannitol, glucose, sorbitol, maltose, dextrates, dextrins and the like.

Water dispersible diluent refers to insoluble pharmaceutical excipients, which disperse readily in water. Examples include microcrystalline cellulose, starch, pre-gelatinized starch, magnesium aluminum silicates and the like.

In one preferred embodiment, the water-soluble diluent is lactose in amounts from about 5% to about 20% by weight.

In another preferred embodiment, the water dispersible diluent is microcrystalline cellulose present in amount from about 5% to about 20% by weight.

The lubricants can be present in the range of about 0.2% to 5% by weight either alone or in combination of total weight of the composition. The lubricants that may be used include talc, stearic acid, magnesium stearate, colloidal silicon dioxide, calcium stearate, zinc stearate, hydrogenated vegetable oil and the like. Preferably, the lubricant is selected from talc, stearic acid, magnesium stearate and colloidal silicon dioxide.

The pharmaceutical composition of the present invention can be prepared by procedures well known to formulation chemists. The method of manufacturing can affect the release characteristics of the composition. The active or their pharmaceutically acceptable hydrates, salts or esters; the hydrophilic polymer of which at least one is carbopol 971P and other one is carbopol 974P; one or more water soluble or water dispersible diluents are either mixed together with lubricants and the blend is directly compressed into tablets or are granulated by compaction followed by sieving and the granules obtained are compressed into tablets.

The above-mentioned process has the advantage over its granulation by aqueous or non-aqueous vehicle used conventionally. Drugs like cefprozil, which are sensitive to moisture and heat, can be effectively processed this way without any difficulty. As the process is devoid of use of any solvents, the potential problem of limiting the residual organic solvent is eliminated.

Hence, the present invention provides a spatial and temporal controlled drug delivery due to advantageously and effectively utilized the semi enteric behavior of carbomers in the acidic environment controlling the initial bursting effect and forming a gel at alkaline pH, thereby controlling the drug release by diffusion.

The details of the invention, its objects and advantages are illustrated hereunder in greater detail in relation to non-limiting exemplary illustrations as per the following examples:

### EXAMPLE 1

| INGREDIENT | WEIGHT (mg/tab) | % W/W |
|---|---|---|
| Cefprozil | 53.5 | 66.3 |
| Carbopol 971P | 21.2 | 2.7 |
| Carbopol 974P | 42.6 | 5.3 |
| Pharmatose DCL21 | 189.5 | 23.7 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cefprozil Released |
|---|---|
| 1 | 38.9 |
| 2 | 71.1 |
| 3 | 77.4 |
| 4 | 79.8 |
| 5 | 83.0 |
| 6 | 87.8 |

### EXAMPLE 2

| INGREDIENT | WEIGHT (mg/tab) | % W/W |
|---|---|---|
| Cefprozil | 530.4 | 66.3 |
| Carbopol 971P | 40.0 | 5.0 |
| Carbopol 974P | 120.0 | 15.0 |
| Pharmatose DCL21 | 93.5 | 11.7 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cefprozil Released |
|---|---|
| 1 | 14.4 |
| 2 | 27.5 |
| 3 | 33.0 |
| 4 | 38.0 |
| 5 | 45.4 |
| 6 | 54.9 |
| 7 | 66.6 |
| 8 | 78.4 |
| 9 | 88.1 |
| 10 | 98.2 |

### EXAMPLE 3

| INGREDIENT | WEIGHT (mg/tab) | % W/W |
|---|---|---|
| Cefprozil | 530.4 | 66.3 |
| Carbopol 971P | 64.0 | 8.0 |
| Carbopol 974P | 96.0 | 12.0 |
| Pharmatose DCL21 | 93.5 | 11.7 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cefprozil Released |
|---|---|
| 1 | 10.9 |
| 2 | 17.6 |
| 3 | 20.6 |
| 4 | 22.4 |
| 5 | 23.8 |
| 6 | 26.3 |
| 7 | 30.3 |
| 8 | 35.1 |
| 9 | 41.0 |
| 10 | 46.3 |

### EXAMPLE 4

| INGREDIENT | WEIGHT (mg/tab) | % W/W |
|---|---|---|
| Cefprozil | 530.4 | 66.3 |
| Carbopol 971P | 50.0 | 6.2 |
| Carbopol 974P | 150.0 | 18.8 |
| Pharmatose DCL21 | 53.5 | 6.7 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cefprozil Released |
|---|---|
| 1 | 14.6 |
| 2 | 25.1 |
| 3 | 30.1 |
| 4 | 34.4 |
| 5 | 40.4 |
| 6 | 47.5 |
| 7 | 56.7 |
| 8 | 66.7 |
| 9 | 78.0 |
| 10 | 88.3 |

### EXAMPLE 5

| INGREDIENT | WEIGHT (mg/tab) | % W/W |
|---|---|---|
| Cefadroxil | 531.6 | 66.5 |
| Carbopol 971P | 40.0 | 5.0 |
| Carbopol 974P | 120.0 | 15.0 |
| Pharmatose DCL21 | 92.4 | 11.6 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cefadroxil Released |
|---|---|
| 1 | 20.5 |
| 2 | 40.9 |
| 3 | 50.3 |
| 4 | 58.0 |
| 5 | 65.0 |
| 6 | 72.4 |
| 7 | 79.2 |
| 8 | 85.1 |
| 9 | 90.2 |
| 10 | 94.5 |

### EXAMPLE 6

| INGREDIENT | WEIGHT (mg/tab) | % W/W |
|---|---|---|
| Cephalexin | 530.2 | 66.3 |
| Carbopol 971P | 40.0 | 5.0 |
| Carbopol 974P | 120.0 | 15.0 |
| Pharmatose DCL21 | 93.8 | 11.7 |
| Magnesium Stearate | 16.0 | 2.0 |
| Total | 800.0 | 100.0 |

Tablets were prepared by direct compression as described earlier and subjected to dissolution studies. These were conducted using USP apparatus-III containing 250 ml of 0.07 N HCl as dissolution media for first 2 hrs followed by pH 6.8 phosphate buffer. The speed was maintained at 5 dips per minute. The dissolution medium was replaced every hour. The cumulative percent drug release from the dosage form is as given hereunder:

| Time (in hrs) | % Cephalexin released |
|---|---|
| 1 | 27.0 |
| 2 | 50.5 |
| 3 | 60.2 |
| 4 | 67.9 |
| 5 | 74.7 |
| 6 | 81.5 |
| 7 | 87.4 |
| 8 | 92.6 |
| 9 | 96.9 |
| 10 | 101.6 |

A bioavailability study was conducted between a controlled released cefprozil formulation, prepared as in example 2, for once daily administration [test formulation (T)] and the immediate release cefprozil product (R) already being marketed, Cefzil® by Bristol Mayers Squibb as a twice daily formulation.

Eight healthy male volunteers were selected for a randomized, biostudy in which each volunteer was administered a single dose (500 mg) of the conventional and two tablets (500 mg each) of cefprozil test formulation tablet with 240 ml of water under fed conditions.

After administration of 500 mg cefprozil conventional dosage form, the blood levels are achieved within 1.5 hour and detectable blood levels are present for 6 hours, whereas long acting modified release formulation according to present invention gave the desired blood levels up to 16 to 18 hours, clearly indicating that it can be used as once daily composition.

The time above MIC achieved by once daily administration of the cefprozil controlled release formulation made in accordance with the present invention are given in Table 1:

TABLE 1

T/MIC (hr.)

| | 0.25 mcg/ml | 2 mcg/ml |
|---|---|---|
| Extended Release Tablet | 17.9 | 11.7 |

When compared with the conventional immediate release formulation, the bioavailability (AUC) and maximum plasma concentraion (Cₘₐₓ) where found to be comparable as given in Table 2 below.

The T>MIC at 0.25 mcg/ml was achieved for about 75% of the dosing interval and T>MIC of 2 mcg/ml was achieved for almost 49% of the dosing interval. Both these values are for a time period of more than the 40% of the dosing interval required indicating that it is an excellent controlled release formulation, which not only achieves the desired pharmacodynamic parameters but also manages to maintain the Cₘₐₓ values substantially similar to those obtained for immediate release formulations. In fact, the Cmax values were within the 80-120% confidence interval recommended by the US FDA.

TABLE 2

| | T/MIC (%) of dosing | | Ratio of | |
|---|---|---|---|---|
| | interval | | Geometric mean | Ratio of |
| | 0.25 | 2 | AUC (0-24) | Cₘₐₓ |
| Product | mcg/ml | mcg/ml | (T/R) | (mcg/ml) |
| REFERENCE | | | | |
| Cefzil Tablet | 78.0 | 43.0 | 103.5 | 112.6 |
| 500 mg (BID) | | | | |
| Immediate | | | | |
| release (Fed) | | | | |
| TEST | | | | |
| (Extended | 74.6 | 48.7 | | |
| Release) | | | | |
| 2 × 500 mg | | | | |
| once-a-day (Fed) | | | | |

AUC was adjusted for the 500 mg immediate release formulation given twice daily using the following formula.

AUC₍₀₋₂₄₎ of controlled release (1000 mg)×AUC₍₀₋₁₂₎ immediate release (500 mg)/2×100%

It is thus possible by way of the present invention to provide a pharmaceutical composition for controlled release of β-lactam antibiotics which would be effective as a once daily dosage form and which would avoid the limitations associated with the compositions disclosed in the prior art.

1. A pharmaceutical composition for controlled drug delivery comprising a β-lactam antibiotic and one or more carbomers.

2. The composition of claim 1 wherein said β-lactam antibiotic is in the form of pharmaceutical acceptable hydrates, salts or esters.

3. The composition of claim 1 wherein said β-lactam antibiotic agent is a cephalosporin.

4. The composition of claim 3 wherein said cephalosporin antibiotics are selected from cefdinir, cefditoren pivoxil, cefepime, cefixime, cefoperazone, cefotetan, cefpodoxine paroxetil, cefprozil, cefazidine, ceftibuten, ceftriaxone, cefuroxime axetil, cephalexin, cefaclor, cefadroxil, cefamandole, cefoxitin, cefalothin, moxalactum, cefapirin, ceftizoxime, cefonicid, cephadrine, loracarbef, cefetamet and pharmaceutically acceptable hydrates, salts or esters thereof.

5. The composition of claim 4 wherein said cephalosporin is cefprozil or its plharinaceutical acceptable hydrates, salts or esters.

6. The composition of claim 5 wherein said cefprozil or their pharmaceutical acceptable hydrates, salts or esters comprise an amount from 100 mg to 1000 mg.

7. The composition of claim 5 wherein said cefprozil or their pharmaceutical acceptable hydrates, salts or ester comprise from about 30-90% w/w of the formulation.

8. The composition of claim 1 wherein said carbomers are a mixture of Carbopol 971P® and Carbopol 974P®.

9. The composition of claim 1 wherein said carbomers comprise about 0.1% to 50% by weight of the controlled release composition.

10. The composition of claim 9 wherein said carbomers are present at a concentration from about 5% to about 50% comprising of Carbopol 971P in an amount from about 0.1% to about 20% by weight and Carbopol 974P in an amount fiom about 0.1% to about 30% by weight of controlled release composition.

11. The composition of claim 1 which further comprises other pharmaceutically acceptable excipients selected amongst water-soluble or water dispersible diluents and lubricants.

12. The composition of claim 11 wherein said water-soluble diluent is selected from lactose, mannitol, glucose, sorbitol, maltose, dextrates, dextrins and the like.

13. The composition of claim 12 wherein said water-soluble diluent is lactose.

14. The composition of claim 13 wherein said lactose amounts from about 5% to about 20% by weight of the formulation.

15. The composition of claim 11 wherein said water dispersible diluent is selected from amongst microcrystalline cellulose, starch, pre-gelatinized starch, magnesium aluminum silicates and the like.

16. The composition of claim 15 wherein said water dispersible diluent is microcrystalline cellulose.

17. The composition of claim 16 wherein said microcrystalline cellulose amounts from about 5% to about 20% by weight of the formulation.

18. The composition of claim 11 wherein said pharmaceutical excipient is either one or a combination of lubricants at a concentration in the range of about 0.2% to 5% by weight of the composition.

19. The composition of claim 11 wherein said lubricant is selected from talc, stearic acid, magnesium stearate, colloidal silicon dioxide, calcium stearate, zinc stearate, hydrogenated vegetable oil and the like.

20. The composition of claim 19 wherein said lubricant is preferably selected from talc, stearic acid, magnesium stearate and colloidal silicon dioxide.

21. The process for the preparation of the pharmaceutical composition comprising mixing together, a β-lactam antibiotic or their pharmaceutically acceptable hydrates, salts or esters; with one or more carbomers and optionally, with one or more water soluble or water dispersible diluents and lubricants to form the blend, and compressing the blend into tablets.

22. The process of claim 21 wherein the blend is compacted into granules.

23. A controlled release composition of β-lactam antibiotic comprising a pharniaceutically effective amount of β-lactam antibiotic, one or more carbomers, a water-soluble and/or water dispersible diluent and pharmaceutically acceptable tablet excipients for controlling the release of β-lactam antibiotic.

24. A controlled release composition comprising a β-lactam antibiotic and a release controlling polymer wherein the Cₘₐₓ is substantially the same as that of a single dose of an immediate release formulation.

25. A controlled release composition of claim 24 wherein the β-lactam antibiotic is cefprozil

26. A controlled release composition comprising a β-lactam antibiotic and a release-controlling polymer wherein the T>MIC at 0.25 mcg/ml was achieved for about 75% of the dosing interval and T>MIC of 2 mcg/inl was achieved for almost 49% of the dosing interval.

27. A controlled release composition of claim 26 wherein the β-lactam antibiotic is cefprozil.

28. A controlled release composition comprising from about 30-90% w/w of cefprozil and from about 0.1-50% by weight of one or a mixture of carbomers and optionally one or more pharmaceutically acceptable excipients selected from amongst diluents and lubricants.

29. A controlled release composition of claim 28 preferably comprising from about 40-80% w/v of cefprozil and from about 0.1-40% w/w of one or a mixture of carbomers and optionally one or more pharmaceutically acceptable excipients selected from amongst diluents and lubricants.
